(19) United States
(12) Patent Application Publication
(10) Pub. No.: US 2025/0249493 A1
(43) Pub. Date: Aug. 7, 2025

(54) METHOD FOR REMOVING POLYCYCLIC AROMATIC HYDROCARBONS WITH BACTERIAL STRAIN

(71) Applicant: KING FAHD UNIVERSITY OF PETROLEUM AND MINERALS, Dhahran (SA)

(72) Inventors: Alexis NZILA, Dhahran (SA); Ajibola Hakeem OKEYODE, Dhahran (SA); Assad AL-THUKAIR, Dhahran (SA); Musa M. MUSA, Dhahran (SA); Mazen Khaled Hamed NAZAL, Dhahran (SA)

(73) Assignee: KING FAHD UNIVERSITY OF PETROLEUM AND MINERALS, Dhahran (SA)

(21) Appl. No.: 18/430,769

(22) Filed: Feb. 2, 2024

(51) Int. Cl. B09C 1/00; C12N 1/20; C12R 1/01

(52) U.S. Cl. CPC B09C 1/00; C12N 1/205; C12N 2500/12; C12N 2523/00; C12R 2001/01

(57) ABSTRACT

A method for removing polycyclic aromatic hydrocarbons from a sample includes contacting the sample with a bacterial strain for a time sufficient to degrade at least one polycyclic aromatic hydrocarbon. The sample includes coronene at a first concentration, and the bacterial strain is Halomonas caseinilytica. The method further includes degrading at least one polycyclic aromatic hydrocarbon and further collecting a product containing coronene at a second concentration that is lower than the first concentration.

### STATEMENT REGARDING PRIOR DISCLOSURE BY THE INVENTORS

Aspects of the present disclosure are described in Nzila, A. et al., “Degradation of the highly complex polycyclic aromatic hydrocarbon coronene by the halophilic bacterial strain Halomonas caseinilytica, 10SCRN4C” published in Volume 49, Number 3, Archives of Environmental Protection, which is incorporated herein by reference in its entirety.

### STATEMENT OF ACKNOWLEDGEMENT

Support provided by the Ministry of Higher Education of Saudi Arabia, through the Deanship of Scientific Research of King Fahd University of Petroleum and Minerals, Saudi Arabia, through project number LS002505 is gratefully acknowledged.

### BACKGROUND

### Technical Field

The present disclosure is directed towards microbial degradation of hydrocarbons and, more particularly, directed to a method for removing polycyclic aromatic hydrocarbons using a bacterial strain.

### Description of Related Art

The “background” description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Polycyclic aromatic hydrocarbons (PAHs) are a group of pollutants found in the environment. They are derived from petroleum products, oil exploration, transport, and exploitation. They may also come from pyrogenic sources, such as the incomplete combustion of organic matter, including wood [Lima, A. L. C., Farrington, J. W. & Reddy, C. M. (2005). Combustion-Derived Polycyclic Aromatic Hydrocarbons in the Environment—A Review. Environ Forensics]. Several side effects have been associated with these compounds, including reduced immune response and reproduction rates in animals, and an increased incidence of cancer [Lee, B. K. & v Vu, T. (2010). “Sources, Distribution and Toxicity of Polyaromatic Hydrocarbons (PAHs) in Particulate Matter,” in Air Pollution].

Removing PAHs from the environment remains a priority. Traditionally, physical, chemical, and biological methods are used to remove these pollutants; however, the biological approach, including biodegradation and/or bioremediation, is a better alternative since it is more environmentally friendly and cheaper than physical and chemical processes. Biodegradation relies on microorganisms that use the PAHs as a carbon source, leading to their removal. PAHs are categorized into two groups based on their number of rings. The first group, known as low molecular weight PAHs (LMW-PAHs), includes two or three ring-containing PAHs. LMW-PAHs include naphthalene, phenanthrene, and anthracene. The second group, known as high molecular weight PAHs (HMW-PAHs), includes PAHs with more than three rings, such as pyrene (four rings), benzopyrene (five rings), and coronene (seven rings). The higher the number of rings, the more resistant the compound is to degradation. As a result, HMW-PAHs, particularly coronene (CRN), tend to accumulate in the environment and have toxic effects on humans, animals, and ecosystems. Currently, only three bacterial strains belonging to the species, Burkholderia cepacia, have been shown to degrade coronene as coronene is one of the most recalcitrant HMW-PAHs and is less amenable to degradation [Juhasz, A. L., Britz, M. L. & Stanley, G. A. (1996). Degradation of high molecular weight polycyclic aromatic hydrocarbons by Pseudomonas cepacia. Biotechnol Lett.].

The B. cepacia strains were initially isolated in the presence of pyrene and were later shown to degrade coronene, but this degradation was only pronounced in the presence of high bacterial cell density or the presence of another substrate (growth-substrate), a process known as co-metabolism. Additionally, one of the strains, which was later reclassified as Stenotrophomonas maltophilia, showed that the degradation of coronene was associated with low toxicity (mutagenicity) against Salmonella typhimurium strains [Juhasz, A. L., Stanley, G. A. & Britz, M. L. (2000). Microbial degradation and detoxification of high molecular weight polycyclic aromatic hydrocarbons by Stenotrophomonas maltophilia strain VUN]. Only one bacterial species, S. maltophilia (formerly known as Burkholderia cepacia), has been reported to degrade coronene and this degradation was performed under low salinity conditions, 0.5% NaCl (w/v).

Although the use of bacterial strains for microbial degradation has been known, each method suffers from certain drawbacks hindering their adoption. Hence, efficient methods must be developed to reduce or eliminate the method limitations. Accordingly, an objective of the present disclosure is directed to a method for removing and/or degrading polycyclic aromatic hydrocarbons across a wide range of temperature and salinity conditions, thereby circumventing the drawbacks of traditional methods.

### SUMMARY

In an exemplary embodiment, a method for removing polycyclic aromatic hydrocarbons from a sample is described. The method includes contacting the sample with a bacterial strain for a time sufficient to degrade at least one polycyclic aromatic hydrocarbon. The sample includes coronene at a first concentration, the bacterial strain is Halomonas caseinilytica. The method further includes degrading the at least one polycyclic aromatic hydrocarbon, and further collecting a product containing coronene at a second concentration lower than the first concentration.

In some embodiments, the bacterial strain is Halomonas caseinilytica, 10SCRN4D.

In some embodiments, bacteria of the bacterial strain are rod-shaped with an average length of 1.0 to 3.0 micrometers (μm) and an average width of 0.1 to 1.0 μm.

In some embodiments, the sample is contacted with a bacterial strain for from 1 to 100 days.

In some embodiments the first concentration is from 10 to 200 micromolar (μM).

In some embodiments, the second concentration is from 20 to 75 percent lower than the first concentration based on the total weight of the sample and the total weight of the coronene before and after the degrading.

In some embodiments, a salinity of the sample is from 0.1 to 25 percent weight sodium chloride by volume based on a total volume of the sample.

In some embodiments, the method includes contacting the sample with the bacterial strain at a temperature from 25 to 50° C.

In some embodiments, the method includes contacting the sample with the bacterial strain at a pH of 4 to 9.

In some embodiments, the method includes contacting the sample with the bacterial strain for 78 to 82 days, and a rate of degradation is 0.100 to 0.130 UM of coronene per day.

In some embodiments, during the contacting the Halomonas caseinilytica reproduces at a doubling rate of 8 to 16 hours.

In some embodiments, the first concentration is 15 to 20 μM, the sample has a pH of 6.8 to 7.2, a temperature of 35 to 40° C., a salinity of 8 to 12 percent weight sodium chloride by volume based on a total volume of the sample. The contacting occurs for 18 to 22 days, and a rate of biodegradation of the coronene is 45 to 50 percent based on an initial concentration of the coronene.

In some embodiments, a starting aliquot of the bacterial strain is 4.5×10⁵ to 5.5×10⁵ colony forming units (CFUs) per milliliter (CFU/mL).

In some embodiments, a maximum count of the bacterial strain is 6×10¹¹ to 8×10¹¹ CFU/mL.

In some embodiments, the first concentration is 15 to 20 μM, the sample has a salinity is 0.2 to 0.8 percent weight sodium chloride by volume based on a total volume of the sample, a pH of 6.8 to 7.2, a temperature of 35 to 40° C., and a rate of biodegradation of the coronene is 55 to 60 percent based on an initial concentration of the coronene.

In yet another exemplary embodiment, the first concentration is 15 to 20 μM, the sample has a temperature of 28 to 32° C., a pH of 6.8 to 7.2, a salinity of 8 to 12 percent weight sodium chloride by volume based on a total volume of the sample, and a rate of biodegradation of the coronene is 40 to 60 percent based on an initial concentration of the coronene.

In some embodiments, the sample further includes one or more polycyclic aromatic hydrocarbons selected from the group consisting of benzo[a]pyrene, pyrene, and phenanthrene.

In some embodiments, the polycyclic aromatic hydrocarbon is benzo[a]pyrene, and 20 to 65 percent of the polycyclic aromatic hydrocarbon is degraded based on an initial amount of the benzo[a]pyrene.

In some embodiments, the polycyclic aromatic hydrocarbon is pyrene, and 35 to 70 percent of the polycyclic aromatic hydrocarbon is degraded based on an initial amount of the pyrene.

In some embodiments, the polycyclic aromatic hydrocarbon is phenanthrene, and 20 to 60 percent of the polycyclic aromatic hydrocarbon is degraded based on an initial amount of the phenanthrene.

These and other aspects of the non-limiting embodiments of the present disclosure will become apparent to those skilled in the art upon review of the following description of specific non-limiting embodiments of the disclosure in conjunction with the accompanying drawings. The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure (including alternatives and/or variations thereof) and many of the attendant advantages thereof will be readily obtained as the same becomes better understood by reference to the following detailed description when considered in connection with the accompanying drawings, wherein:

FIG. 1A is a schematic flow chart of a method for removing polycyclic aromatic hydrocarbons from a sample, according to certain embodiments;

FIG. 1B is a scanning electron microscopy (SEM) image of 10SCRN4D bacterial strain, according to certain embodiments;

FIG. 1C depicts growth profile of Halomonas caseinilytica, 10SCRN4D strain as a function of coronene (CRN) concentration, according to certain embodiments;

FIG. 1D depicts doubling time (dt) of Halomonas caseinilytica, 10SCRN4D strain as a function of CRN concentration, according to certain embodiments;

FIG. 2 depicts the quantification of CRN degradation by Halomonas caseinilytica, 10SCRN4D strain using gas chromatography (GC), according to certain embodiments;

FIG. 3A depicts the growth profile of Halomonas caseinilytica, 10SCRN4D strain as a function of temperature, according to certain embodiments;

FIG. 3B depicts the effect of temperature on the ability of Halomonas caseinilytica, 10SCRN4D strain to degrade CRN, according to certain embodiments;

FIG. 4A depicts the growth profile of Halomonas caseinilytica, 10SCRN4D strain as a function of salinity at different sodium chloride (NaCl) concentrations, according to certain embodiments;

FIG. 4B depicts the effect of salinity on the ability of Halomonas caseinilytica, 10SCRN4D strain to degrade CRN, according to certain embodiments; and

FIG. 5 depicts the ability of Halomonas caseinilytica, 10SCRN4D strain to degrade CRN over a time period of 80 days, according to certain embodiments.

### DETAILED DESCRIPTION

In the following description, it is understood that other embodiments may be utilized, and structural and operational changes may be made without departure from the scope of the present embodiments disclosed herein.

Reference will now be made to specific embodiments or features, examples of which are illustrated in the accompanying drawings. In the drawings, whenever possible, corresponding or similar reference numerals will be used to designate identical or corresponding parts throughout the several views. Moreover, references to various elements described herein are made collectively or individually when there may be more than one element of the same type. However, such references are merely exemplary in nature. It may be noted that any reference to elements in the singular may also be constructed to relate to the plural and vice-versa without limiting the scope of the disclosure to the exact number or type of such elements unless set forth explicitly in the appended claims.

When describing the present disclosure, the terms used are to be construed in accordance with the following definitions, unless a context dictates otherwise.

Embodiments of the present disclosure will now be described more fully hereinafter with reference to the accompanying drawings wherever applicable, in that some, but not all embodiments of the disclosure are shown.

As used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

The use of the terms “include,” “includes,” “including,” “have,” “has,” or “having” should be generally understood as open-ended and non-limiting unless specifically stated otherwise.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may include certain elements or features does not exclude other embodiments of the present disclosure that do not contain those elements or features.

The term “aromatic compounds” or “aromatic rings,” as used herein, refers to chemical compounds that have conjugated planar hydrocarbon rings that, by the theory of Hückel, have a cyclic, delocalized (4n+2) pi-electron system. Non-limiting examples of aromatic compounds include benzene, benzene derivatives, compounds having at least one benzene ring in their chemical structure, toluene, ethylbenzene, p-xylene, m-xylene, mesitylene, durene, 2-phenylhexane, biphenyl, phenol, aniline, nitrobenzene, and the like.

Aspects of the present disclosure are directed to a method for biodegradation of polyaromatic hydrocarbons (PAHs) using a bacterial strain Halomonas caseinilytica, 10SCRN4D. This strain was found to be effective in degrading coronene, a high molecular weight PAH, and other smaller molecular weight PAHs, such as benzo[a]pyrene, pyrene, and phenanthrene, across a wide range of temperature and salinity conditions, thus highlighting the potential of this strain in bioremediation to degrade PAHs.

PAHs may include, but are not limited to, naphthalene, acenaphthene, acenaphthylene, anthracene, fluoranthene, fluorene, chrysene, phenanthrene, pyrene, coronene, chrysene, ovalene, dibenz(a,h)acridine, benz[a]anthracene, benzo[a]pyrene, benzo[e]pyrene, benzo[b]fluoranthene, benzo[g,h,i]perylene, benzo[j]fluoranthene, benzo[k]fluoranthene, dibenzo[a,h]anthracene, indeno[1,2,3-c,d]pyrene, and the like.

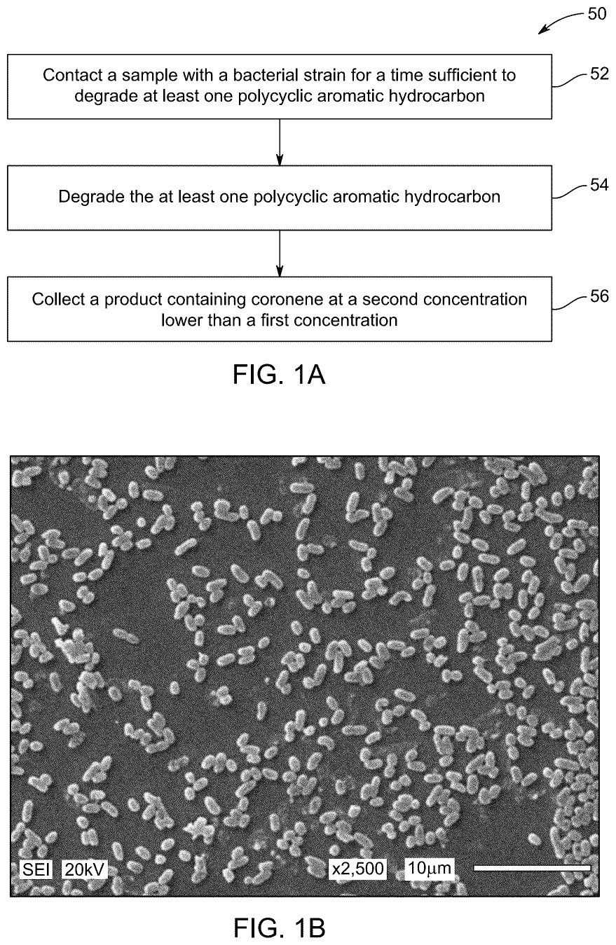

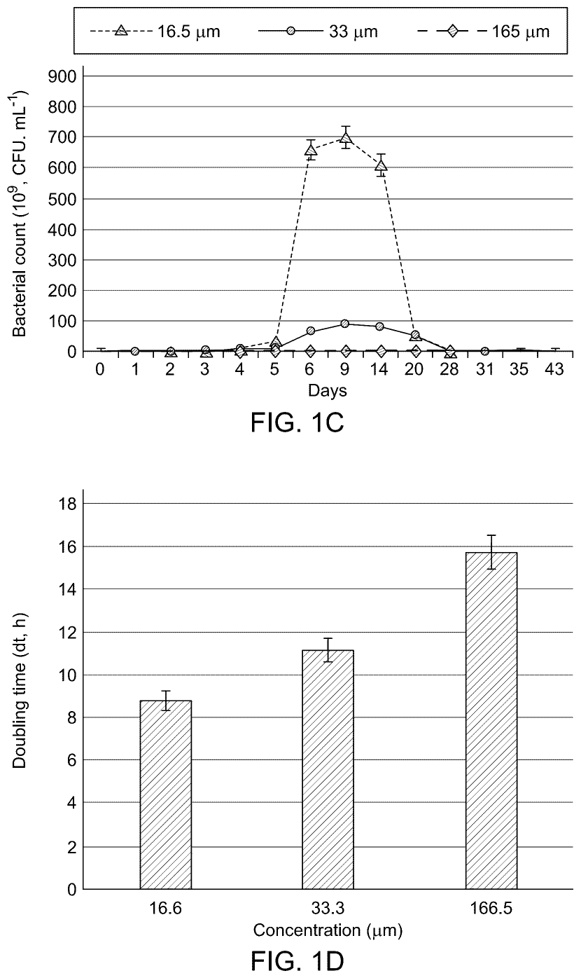

FIG. 1A illustrates a schematic flow chart of a method 50 for removing polycyclic aromatic hydrocarbons (PAHs) from a sample. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes contacting the sample with a bacterial strain for a time sufficient to degrade at least one polycyclic aromatic hydrocarbon (PAH). The sample may include groundwater or any other liquid contaminated with one or more PAHs. The sample may be collected from soil or sludge. In some embodiments, the groundwater or contaminated liquid may further include crude oil, diesel fuel, lubricating oil, or organic compounds like octane, decane, hexadecane, methyl cyclopentane, methylcyclohexane, heptamethylnonane, benzene, toluene, ethylbenzene, m-, o- and p-xylenes, and the like. In some embodiments, the sample includes one or more PAHs selected from benzo[a]pyrene, pyrene, and phenanthrene. In a specific embodiment, the sample includes coronene. The coronene is present at a first concentration in a range of 10 to 200 micromolar (μM), preferably 15 to 190 μM, preferably 20 to 180 μM, preferably 25 to 170 μM, preferably 30 to 160 μM, preferably 40 to 150 μM, preferably 50 to 140 μM, preferably 60 to 130 μM, preferably 70 to 120 μM, preferably 80 to 110 μM, preferably 90 to 100 μM, and more preferably about 16.5 μM, about 33 μM, and about 165 μM.

In some embodiments, the salinity of the sample is in the range of 0.1 to 15%, preferably 0.5 to 14%, preferably 1 to 13%, preferably 2 to 12%, preferably 4 to 11%, preferably 5 to 10%, preferably about 0.5%, more preferably about 5%, and yet more preferably about 10% weight sodium chloride by volume based on the total volume of the sample.

The sample is contacted with a bacterial strain for degradation of PAHs. The bacterial strain belongs to Halomonas genus, and more specifically, is at least one selected from Halomonas alkaliantarctica, Halomonas alkaliphile, Halomonas axialensis, Halomonas boliviensis, Halomonas campaniensis, Halomonas canadensis, Halomonas caseinilytica, Halomonas cupida, Halomonas daqingensis, Halomonas denitrificans, Halomonas desiderata, Halomonas elongate, Halomonas eurihalina, Halomonas halmophila, Halomonas halocynthiae, Halomonas halodenitrificans, Halomonas hamiltonii, Halomonas ilicicola, Halomonas indalinina, Halomonas israelensis, Halomonas jeotgali, Halomonas johnsoniae, Halomonas magadiensis, Halomonas eptunia, Halomonas pacifica, Halomonas saliphila, Halomonas sinaiensis, Halomonas smyrnensis, Halomonas stevensii, Halomonas sulfidaeris, Halomonas titanicae, Halomonas ventosae, Halomonas venusta, Halomonas vilamensis, and the like. In a preferred embodiment, the bacterial strain is Halomonas caseinilytica, more specifically, Halomonas caseinilytica, 10SCRN4D. In some embodiments, the bacterial strain may be a combination of one or more strains from the Halomonas genus. In an embodiment, the bacterial strain is a halophilic bacterial strain.

Bacteria in the bacterial strain are rod-shaped with an average length of 1.0 to 3.0 micrometers (μm), preferably 1.1 to 2.9 μm, preferably 1.2 to 2.8 μm, preferably 1.3 to 2.7 μm, preferably 1.4 to 2.6 μm, preferably 1.5 to 2.5 μm, preferably 1.6 to 2.4 μm, preferably 1.7 to 2.3 μm, preferably 1.8 to 2.2 μm, preferably 1.9 to 2.1 μm, and preferably about 2.0 μm, and an average width of 0.1 μm to 2.0 μm, preferably 0.2 to 1.9 μm, preferably 0.3 to 1.8 μm, preferably 0.4 to 1.7 μm, preferably 0.5 to 1.6 μm, preferably 0.6 to 1.5 μm, preferably 0.7 to 1.4 μm, preferably 0.8 to 1.3 μm, preferably 0.9 to 1.2 μm, and preferably 1.0 to 1.1 μm. In some embodiments, the bacteria are predominantly rod-shaped with an average length of 1.0 to 3.0 μm, more preferably 2.1 μm and an average width of 0.1 to 1.0 μm, more preferably 0.5 μm. In some embodiments, the bacteria are rod-shaped, sphere-shaped, oblong-shaped, rectangular-shaped, oval-shaped, and the like. In some embodiments, the Halomonas caseinilytica, 10SCRN4D is gram positive.

The bacterial strain belonging to the Halomonas genus, specifically Halomonas caseinilytica, 10SCRN4D, is contacted with the PAHs for a sufficient period to degrade the PAHs. In some embodiments, the sample is contacted with a bacterial strain for 1 to 100 days, preferably 1 to 95 days, preferably 5 to 90 days, preferably 10 to 85 days, preferably 15 to 80 days, preferably 20 to 75 days, preferably 25 to 70 days, preferably 30 to 65 days, preferably 35 to 60 days, preferably 40 to 55 days, and preferably 45 to 50 days. In some embodiments, the sample is contacted with the bacterial strain, preferably for about 80 days. In some embodiments, the sample is contacted with a bacterial strain at a temperature range of about 25 to 50° C., preferably 30 to 45° C., preferably 35 to 40° C.; and at a pH of 4 to 9, preferably 5 to 8, preferably 6 to 7, and yet more preferably at about 7. In some embodiments, the sample is contacted with a bacterial strain, Halomonas caseinilytica, for about 78 to 82 days and the rate of degradation of coronene is in the range of 0.100 to 0.130 UM of coronene per day. In a specific embodiment, the sample, when contacted with the bacterial strain Halomonas caseinilytica for 80 days, the rate of degradation of coronene is about 0.200 μM of coronene per day.

At defined parameters for the first concentration, salinity, contact time, temperature, pH, and choice of PAH, the bacterial strain, Halomonas caseinilytica, reproduces at a doubling rate of about 8 to 16 hours, preferably 10 to 14 hours, and more preferably 11 to 13 hours, and yet more preferably about 12 hours.

At step 54, the method 50 includes degrading the at least one polycyclic aromatic hydrocarbon. The degradation of the PAHs is dependent on several factors such as temperature, salinity, the first concentration of the sample, pH, contact time, bacterial load, and the like. In some embodiments, when an initial concentration of the coronene is 15 to 20 μM, the sample has a pH of 6.8 to 7.2, a temperature of 35 to 40° C., a salinity of 8 to 12 percent weight sodium chloride by volume based on a total volume of the sample, and a contact time of 18 to 22 days, the rate of biodegradation of the coronene is 45 to 50 percent based on the initial concentration of the coronene. In some embodiments, when an initial concentration of the coronene is 15 to 20 μM, more preferably 16.6 μM, the sample has a pH of 6.8 to 7.2, more preferably 7.0, a temperature of 35 to 40° C., more preferably 37° C., a salinity of 8 to 12 percent, more preferably 10 percent, weight sodium chloride by volume based on a total volume of the sample, and a contact time of 18 to 22 days, more preferably 20 days, the rate of biodegradation of the coronene is 45 to 50 percent, more preferably 48 percent, based on the initial concentration of the coronene.

In some embodiments, a starting aliquot of the bacterial strain is 4.5×10⁵ to 5.5×10⁵ colony forming units (CFUs) per milliliter (CFU/mL). In some embodiments, a starting aliquot of the bacterial strain is 4.5×10⁵ to 5.5×10⁵ CFU/mL, preferably 4.6×10⁵ to 5.4×10⁵ CFU/mL, preferably 4.7×10⁵ to 5.3×10⁵ CFU/mL, preferably 4.8×10⁵ to 5.2×10⁵ CFU/mL, preferably 4.9×10⁵ to 5.1×10⁵ CFU/mL, and more preferably about 5.0×10⁵ CFU/mL. In some embodiments, the maximum count of the bacterial strain is 6×10¹¹ to 8×10¹¹ CFU/mL. In some embodiments, the maximum count of the bacterial strain is 6×10¹¹ to 8×10¹¹ CFU/mL, preferably 6.1×10¹¹ to 7.9×10¹¹ CFU/mL, preferably 6.2×10¹¹ to 7.8×10¹¹ CFU/mL, preferably 6.3×10¹¹ to 7.7×10¹¹ CFU/mL, preferably 6.4×10¹¹ to 7.6×10¹¹ CFU/mL, preferably 6.5×10¹¹ to 7.5×10¹¹ CFU/mL, preferably 6.6×10¹¹ to 7.4×10¹¹ CFU/mL, preferably 6.7×10¹¹ to 7.3×10¹¹ CFU/mL, preferably 6.8×10¹¹ to 7.2×10¹¹ CFU/mL, preferably 6.9×10¹¹ to 7.1×10¹¹ CFU/mL, and more preferably about 7×10¹¹ CFU/mL.

In a specific embodiment, when the first concentration is 15 to 20 μM, the sample has a salinity of 0.2 to 0.8 percent weight sodium chloride by volume based on the total volume of the sample, a pH of 6.8 to 7.2, a temperature of 35 to 40° C., the rate of biodegradation of the coronene is 55 to 60 percent based on an initial concentration of the coronene. In some embodiments, when the first concentration is 15 to 20 μM, more preferably 16.5 μM, the sample has a salinity of 0.2 to 0.8, more preferably 0.5, percent weight sodium chloride by volume based on the total volume of the sample, a pH of 6.8 to 7.2, more preferably 7.0, a temperature of 35 to 40° C., more preferably 37° C., the rate of biodegradation of the coronene is 55 to 60 percent, more preferably 57 percent, based on an initial concentration of the coronene.

In some embodiments, when the first concentration is 15 to 20 μM, the sample has a temperature of 28 to 32° C., a pH of 6.8 to 7.2, a salinity of 8 to 12 percent weight sodium chloride by volume based on a total volume of the sample, the rate of biodegradation of the coronene is 40 to 60 percent based on an initial concentration of the coronene. In some embodiments, when the first concentration is 15 to 20 μM, more preferably 16.5 μM, the sample has a temperature of 28 to 32° C., more preferably 30° C., a pH of 6.8 to 7.2, more preferably 7.0, a salinity of 8 to 12 percent, more preferably 10 percent, weight sodium chloride by volume based on a total volume of the sample, the rate of biodegradation of the coronene is 40 to 60 percent, more preferably 50 percent, based on an initial concentration of the coronene.

At step 56, the method 50 includes collecting a product containing coronene at a second concentration lower than the first concentration. In some embodiments, the second concentration is 20 to 75 percent lower than the first concentration based on the total weight of the sample and the total weight of the coronene before and after the degrading. In some embodiments, at least 20 percent, preferably at least 25 percent, preferably at least 30 percent, preferably at least 35 percent, preferably at least 40 percent, preferably at least 45 percent, preferably at least 50 percent, preferably at least 55 percent, preferably at least 60 percent, preferably at least 65 percent, preferably at least 70, and more preferably about 75 percent lower than the first concentration based on the total weight of the sample and the total weight of the coronene before and after the degradation.

In some embodiments, the PAH is benzo[a]pyrene and 20 to 65 percent of benzo[a]pyrene is degraded based on an initial amount of benzo[a]pyrene. In some embodiments, the PAH is benzo[a]pyrene and at least about 20 percent, preferably at least 25 percent, preferably at least 30 percent, preferably at least 35 percent, preferably at least 40 percent, preferably at least 45 percent, preferably at least 50 percent, preferably at least 55 percent, preferably at least 60 percent, and preferably about 65 percent of benzo[a]pyrene is degraded based on an initial amount of the benzo[a]pyrene. In a preferred embodiment, after 30 days, when the PAH is benzo[a]pyrene, the bacterial strain degrades about 43.68%±19.36% of benzo[a]pyrene based on the initial amount of the benzo[a]pyrene at 37° C., a pH of 7, and 10% NaCl in the presence of 20 UM benzo[a]pyrene.

In some embodiments, the PAH is pyrene and 35 to 70 percent of pyrene is degraded based on an initial amount of the pyrene. In some embodiments, the PAH is pyrene and at least about 35 percent, preferably at least 40 percent, preferably at least 45 percent, preferably at least 50 percent, preferably at least 55 percent, preferably at least 60 percent, preferably at least 65 percent, and more preferably about 70 percent of pyrene is degraded based on an initial amount of the pyrene. In a preferred embodiment, after 30 days, when the PAH is pyrene, the bacterial strain degrades about 51.75%±13.67% of pyrene based on the initial amount of the pyrene at 37° C., a pH of 7, and 10% NaCl in the presence of 20 μM pyrene.

In some embodiments, the PAH is phenanthrene and 20 to 60 percent of phenanthrene is degraded based on an initial amount of the phenanthrene. In some embodiments, the PAH is phenanthrene and at least about 20 percent, preferably at least about 25 percent, preferably at least about 30 percent, preferably at least about 35 percent, preferably at least about 40 percent, preferably at least about 45 percent, preferably at least about 50 percent, preferably at least about 55 percent, and more preferably about 60 percent of phenanthrene is degraded based on an initial amount of the phenanthrene. In some embodiments, after 30 days, when the PAH is phenanthrene, the bacterial strain degrades about 41.83%±16.24% of the phenanthrene based on the initial amount of the phenanthrene at 37° C., a pH of 7, and 10% NaCl in the presence of 20 μM phenanthrene.

### EXAMPLES

The following examples describe and demonstrate exemplary embodiments of the method for removing polycyclic aromatic hydrocarbons (PAH) using bacterial strains. The examples are provided solely for the purpose of illustration and are not to be construed as limitations of the present disclosure, as many variations thereof are possible without departing from the spirit and scope of the present disclosure.

### Example 1: Materials

The following chemicals, coronene (CRN), benzo[a]pyrene, pyrene, anthracene, magnesium sulfate (MgSO₄), calcium chloride (CaCl₂)), monopotassium phosphate (KH₂PO₄), dipotassium phosphate (K₂HPO₄), ammonium nitrate (NH₄NO₃), ferric chloride (FeCl₃), and agar used for the preparation of culture media were all procured from Sigma-Aldrich (St. Louis, MO, USA). CRN and the other PAHs had a purity of ≥96%. Chemicals for Luria-Bertani (LB) medium, including tryptone, yeast extract, agar, and common salt (NaCl), were purchased from Difco (Detroit, MI, USA). The solvents chloroform and ethyl-acetate were of analytical grade and purchased from Sigma-Aldrich (St. Louis, MO, USA).

### Example 2: Sample Collection

Contaminated soil samples were collected from the filling station at King Fahd University of Petroleum and Minerals (KFUPM), Saudi Arabia. The filling station (location, 26.309144410341673, 50.13994252761753) serves as a large fuel depot for school buses within the KFUPM campus. It is known for its oil-contaminated soils, which is an environment to isolate bacteria capable of degrading PAHs.

### Example 3: Enrichment Cultures

About 1.0 g of the soil sample was enriched with a 100 mL Bushnell-Hass (BH) culture medium, which consisted of 0.2 g MgSO₄, 0.02 g CaCl₂), 1.0 g KH₂PO₄, 1.0 g K₂HPO₄, 1.0 g NH₄NO₃, 0.05 g FeCl₃, at 10% and 15% weight by volume (w/v) of sodium chloride (NaCl) per liter, and the final suspension was adjusted to a pH of 7.0. This suspension was then supplemented with 25 mg⁻¹ (82.5 μM) of CRN as the sole carbon source before culturing it at 37° C. at 120 rotations per minute (rpm). After 3 to 4 weeks, the culture was transferred to a fresh culture medium (1/10, v/v) for another 2 to 3 weeks, and this transfer was repeated 3 to 4 times until the growth of bacteria was observed. Bacterial colonies were separated using solid agar culture, prepared in BH-medium (1%, w/v), and then incubated at 37° C. for 15 to 21 days. The purity of the isolated individual colonies was confirmed by another solid agar culture (in the same conditions), and after that, these colonies were cryopreserved in 15% volume by volume (v/v) glycerol.

### Example 4: Bacterial Enumeration

Bacteria were enumerated or counted following serial dilutions of cultures by 10-, 100-, and 1000-fold in phosphate buffer (PBS, pH 7) containing NaCl (10% w/v). Around 0.1 mL of the diluted culture was then spread onto solid agar plates prepared in rich medium LB containing NaCl (10% w/v). After that, the cultures were kept for 24 hours (h) at 37° C., the colony numbers were estimated, and the results were presented as colony-forming units per mL (CFU·mL-1).

### Example 5: Scanning Electron Microscopy (SEM)

About 1.0 mL of an aliquot of bacterial cells was cultured in 10 mL of LB medium in a shaker incubator at 37° C. and 120 rpm for 12 hours. The bacterial cells were then centrifuged at 3500×g for 5 minutes (min) at 4° C. to harvest the cells. The resulting cells were then suspended in 1 mL of phosphate buffer (PBS, pH 7). 20 μL of 25% glutaraldehyde solution (v/v), the fixative agent, was added to the suspension and was incubated for 12 h at 25° C. After, around 100 μL of the suspension was fixed by spreading it on microscopic glass slides and incubating at 37° C. for another 12 h. To dehydrate the bacterial cells, the slides were embedded in a serial dilution of ethanol, 10%, 25%, 50%, 75%, 96%, and 100% (v/v). The slides were then coated with gold for 5 minutes before being observed using a JSM-T300 scanning electron microscope (JEOL, Japan).

### Example 6: Bacteria Species Identification

For bacterial species identification by 16S rRNA gene sequencing and phylogeny, the cryopreserved bacterial cells were pre-cultured in a 100 mL LB medium and centrifuged at 6000×g for 5 minutes at 4° C. to harvest the bacterial cells. The cells were lysed, followed by DNA extraction and purification using a Qiagen Powerfecal Kit (Qiagen, Hilden, Germany). Then, the 16S rRNA gene was amplified and sequenced. The sequence analysis was carried out using “the BLAST” software tools available at the National Center for Biotechnology Information, NCBI. The program compares nucleotide sequences to those present in this database and calculates the statistical significance. In addition, the program will list the phylogenetically related sequences based on their statistical significance.

Experiments were initiated with the preculturing of the bacterial strain in LB medium, and around 5×10⁵ CFUs of bacteria were then cultured in 50 mL BH medium to test the effect of temperature, pH, and salinity on the degradation of CRN. More specifically, bacterial growth was tested in the presence of 16.5 μM, 33 μM, and 165 μM of CRN, initially dissolved in dimethyl sulfoxide (DMSO) at the temperatures of 30° C., 35° C., 40° C., and 45° C. Bacterial growth was also tested at a salinity of 0%, 5%, 10%, 15%, and 20% of NaCl (w/v), and pHs of 5, 6, 7, and 8. The ability of bacteria to grow in these various conditions was assessed by counting colonies and quantifying the remaining CRN in the culture. The ability of this strain to degrade the PAHs benzo[a]pyrene, pyrene, and anthracene separately was also evaluated. Cultures were initiated with 5×10⁵ CFUs of bacterial cells in BH-medium in the presence of 25 μM of each of the three tested PAHs. The cultures were initially dissolved in DMSO and separately cultured in 100 mL BH medium at 37° C., with a salinity of 10% and pH of 7 for 30 days.

### Example 8: Quantification of CRN and Other PAHs by Gas Chromatography (GC)

CRN was quantified following a 100 mL culture of the bacteria in the presence of CRN at various conditions. After each culture, 100 mL samples were collected, and the remaining CRN was first sonicated for 30 minutes and then extracted twice, using 50 mL of ethyl acetate. The resulting organic layer was dehydrated using sodium sulfate before drying under a vacuum. The remaining residue (pellet) was then dissolved in 500 μL of chloroform before analysis in GC, as described below. The same protocol for pellet isolation was also employed for the other PAHs (Benzo [a]pyrene, pyrene, and anthracene); however, the quantification procedure for CRN is different from that used for the other PAHs, as described below.

A multi-reaction monitoring method (MRM) was developed to detect and quantify CRN using GC coupled with a tandem mass spectrometer (Shimadzu GC-MS/MS TQ8030, Japan). A Rxi 5 Sil MS capillary column (30 m×id 0.25 mm×ft 0.25 μm) (Restek, USA, Sr.No: 1652241) was used to separate CRN. High-purity helium (99.999%) gas was used as the carrier gas with a flow rate of 2 mL/min and a total run time of 17.5 min. The extract (1.0 μL) was injected using split-less mode. The injector temperature was maintained at 320° C. The column oven temperature was ramped from 200° C. after a holding time of 1 min to 300° C. at a rate of 15° C./min with a holding time of 10 min. The temperature of the detector was set at 280° C. The precursor's mass of CRN was 300.1, and its detection mass ion and the collision energy for this transition were 150.10 m/z and 15 electron volts (eV), respectively. A 5-point standard curve of CRN (0, 0.15, 0.3, 1.5, and 3 μmol L⁻¹) was used to assess the concentration. The percentage of remaining CRN was evaluated according to the formula: % degradation CRN=[(C−T)/C]*100, where C is the coronene concentration in the control sample (without bacteria) and T is coronene concentration in the tested sample (with bacteria).

As previously reported, the quantification of benzo[a]pyrene, pyrene, and anthracene was done using GC with a flame ionization detector. This method used an HP-5 column (30 m×id 0.32 mm). The initial oven temperature was 120° C. for 2 minutes, which increased to 250° C. at 11° C./minute, then was held for 50 minutes. The injector and detector temperatures were 310° C. and 320° C., respectively. The helium flow rate, the injected volume, and the split ratio were 15 milliliters per minute inverse (mL min⁻¹), 1.0 μL, and 10:1, respectively. The same formula, CRN=[(C−T)/C]*100, was used to quantify the remaining PAHs. All these experiments were performed in duplicate. Multi-point calibration curves were established in the range starting from 0.010 to 1.000 mg/L with a squared correlation coefficient >0.9.

### Example 9: Statistical Analysis

The data were analyzed using a one-way analysis of variance (ANOVA), student's t-test, and linear regression fitting model of the R software packages. The linearity of the data was determined using Pearson's correlation coefficient, and the significance level in all tests was set at a threshold of p<0.05.

### Example 10: Enrichment, Strain Isolation, and Species Identification

The enrichment experiments were carried out at 10% and 15% NaCl in the presence of CRN as the sole carbon source. Growth, as determined by turbidity, was observed at 10% but not 15% NaCl. Two bacterial colonies were identified by direct visualization after spreading the culture on an agar plate. However, using a light microscope at 1000 times magnification (1000×), both colonies were observed to be rod-shaped and punctiform, with convex elevation, spherical, light yellow, and an entire margin. Thus, the colonies were identical and named 10SCRN4D.

Further studies have shown that this bacterial strain, 10SCRN4D, is gram-positive, and SEM confirmed that 10SCRN4D is rod-shaped with an average size of 2.1 micrometers (μm) in length and 0.5 μm in width, as shown in FIG. 1B. The 16S rRNA gene sequencing, followed by the BLAST homology analysis of available 16S rRNA gene sequences in the National Center of Biotechnology Information (NCBI) database, showed that this strain belongs to the Halomonas caseinilytica species, based on the 99.93% homology with Halomonas caseinilytica strain NY-7 16S ribosomal RNA gene (NCBI, gene bank ref: OP815347.1). Halomonas genus consists of halophilic (salt-tolerant) bacterial species that grow in a salinity range of 5% to 20% NaCl. This genus is popular as a host for microbial cell factory engineering due to its fast growth in high salt and pH conditions, leading to the absence of contamination during fermentation processes without the need for sterilization [Xiao-Ran, J., Jin, Y., Xiangbin, C. & Guo-Qiang, C. (2018). “Chapter Eleven-Halomonas and Pathway Engineering for Bioplastics Production,” in Methods in Enzymology, ed. N. Scrutton, which is incorporated herein by reference in its entirety].

Concerning pollutant biodegradation, several species of Halomonas have been shown to degrade PAHs. For instance, degradation of naphthalene, pyrene, or benzo[a]pyrene has been reported in Halomonas sp., Halomonas shengliensis, and Halomonas smyrnensis [Budiyanto, F., Thukair, A., Al-Momani, M., Musa, M. M. & Nzila, A. (2018). Characterization of Halophilic Bacteria Capable of Efficiently Biodegrading the High-Molecular-Weight Polycyclic Aromatic Hydrocarbon Pyrene, which is incorporated herein by reference in its entirety]. The biodegradation of naphthalene has also been shown in Halomonas pacifica [Cheffi, M., Hentati, D., Chebbi, A., Mhiri, N., Sayadi, S., Marqués, A. & Chamkha, M. (2020). Isolation and characterization of a newly naphthalene-degrading Halomonas pacifica, strain Cnaph3: biodegradation and biosurfactant production studies, which is incorporated herein by reference in its entirety]. The monoaromatic compounds phenol, catechol, and para-aminoacetanilide have been demonstrated to be degraded in Halomonas campisalis and Halomonas sp. TBZ3 [Alva, V. A. & Peyton, B. M. (2003). Phenol and Catechol Biodegradation by the Haloalkaliphile Halomonas campisalis: Influence of pH and Salinity, which is incorporated herein by reference in its entirety].

### Example 11: Effect of Concentration of CRN

PAHs are toxic to living organisms and bacteria. To evaluate the extent of this toxicity, the 10SCRN4D strain was grown in the presence of increasing concentrations of CRN (16.6 μM, 33 μM, and 166.5 μM), a pH of 7, a temperature of 37° C., and a salinity of 10% NaCl. Initially, the effect of DMSO (0.1% v/v) was assessed by monitoring the growth of a culture of around 10⁵ CFU mL⁻¹ 10SCRN4D for 30 days. No bacterial growth was observed; thus, these bacteria may not utilize DMSO as their carbon source. As a result, in all subsequent studies, CRN was dissolved in DMSO as a neutral solvent.

FIG. 1C shows bacterial growth in the presence of 16.6 μM, 33.3 μM, and 166.5 μM of CRN as a function of time for 30 days. Starting from an aliquot of around 5×10⁵ CFU mL-1, the desired growth was reached within 9 days for the three tested concentrations. However, the maximum counts differed. At the lowest concentration of about 16.6 μM CRN, the maximum count was around 7×10¹¹ CFU mL-1, while this value decreased to about 1×10¹¹ CFU mL-1 at 33.3 μM CRN. The lowest maximum count of about 1×10¹⁰ CFU mL-1 was observed with the highest concentration of CRN (166.5 μM). All these observations were supported by the computation of the doubling times (dt), with values of 8.78 hours, 11.13 hours, and 15.71 hours at 16.6 μM, 33.3 μM, and 166.5 μM of CRN, respectively, as can be seen from FIG. 1D. The difference of dt values pertaining to these 3 concentrations was statistically significant (ANOVA test, p<0.05) and the trend analysis indicated a linear relationship equation of dt=0.001*C+0.37 (R²=0.93, p-value<0.05, where C is the CRN concentration).

The effect of CRN concentration was also assessed by quantifying the remaining CRN using the GC technique. Before the analysis, a 5-point CRN concentration curve (0 μM, 0.15 μM, 0.3 μM, 1.5 μM, and 3 μM) was quantified. The data was plotted on a linear x and y graph, and the results showed a squared correlation coefficient of R²=0.9996, which was used as the standard curve for determining unknown CRN concentrations.

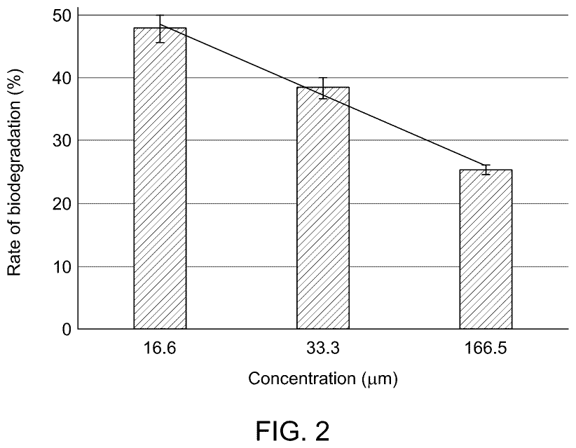

As shown in FIG. 2, the degradation of CRN decreases as the concentration of CRN used in the medium increases. For instance, after 20 days, at 16.6 μM the degradation rate was 48%, and this value decreased to 38% and 25% at 33.3 and 165 μM, respectively, an illustration of the toxicity of the CRN. The ANOVA test showed that these values were statistically different (p<0.05), while the trend analysis indicates a quadratic model according to the equation:

Degradation (%)=0.0013*C²−0.27*C+52 (Adj=0.8618,p-value<0.05).

These results, showing the decrease of bacterial growth as CRN concentrations increase, are consistent with previously reported studies using other PAHs. For instance, a reduction in growth was observed with the increasing anthracene concentration in Bacilluslicheniformis, Ochrobactrum sp., and a co-culture of Ralstonia pickettii and Thermomonas haemolytica [Arulazhagan, P. & Vasudevan, N. (2011). Biodegradation of polycyclic aromatic hydrocarbons by a halotolerant bacterial strain Ochrobactrum sp. VA1, which is incorporated herein by reference in its entirety], phenanthrene in a co-culture of Pseudomonas citronellolis and S. maltophilia [Nzila, A., Sankara, S., Al-Momani, M., Musa Musa, M. & Musa, M. M. (2017). Isolation and characterization of bacteria degrading polycyclic aromatic hydrocarbons: phenanthrene and anthracene, which is incorporated herein by reference in its entirety], and pyrene in Ochrobactrum sp., Achromobacter xylosoxidans, and in the halophilic strains of Halomonas shengliensis and Halomonas smyrnensis.

### Example 12: Temperature Effect

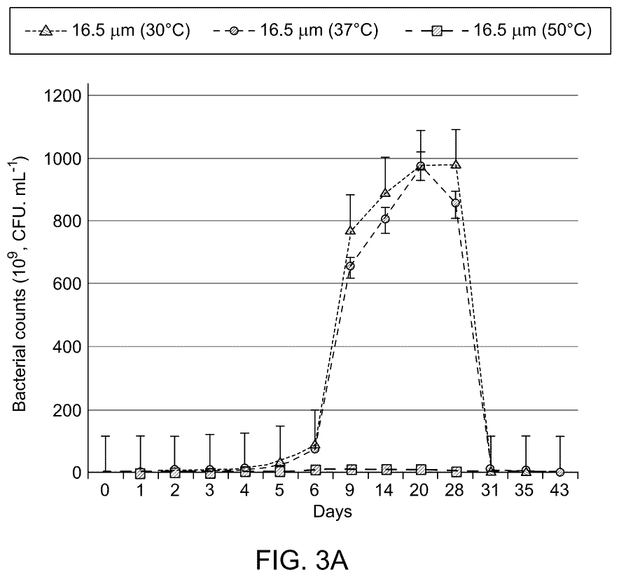

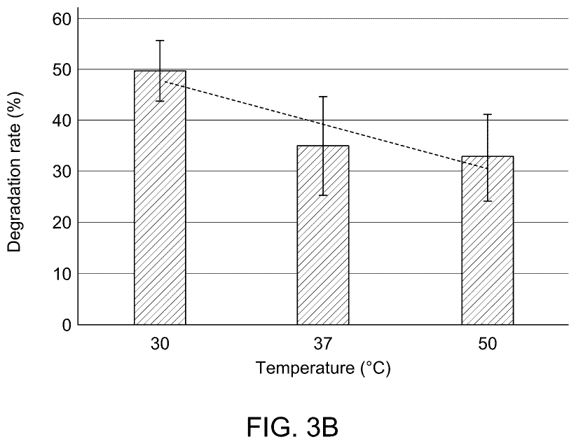

In relation to the temperature effect, the growth of the 10SCRN4D strain was monitored at 30° C., 37° C., and 50° C. while keeping the CRN concentration, pH, and salinity fixed at 16.6 μM, 7, and 10% of NaCl, w/v, respectively. The highest growth rate was observed at 30° C. and 37° C., with the culture reaching a maximum count of around 1012 CFU mL-1 on day 20 at 37° C. and on day 28° C. at 30° C., as shown in FIG. 3A. In comparison, at 50° C., bacterial counts remained below 109 CFU mL-1 throughout the experimental period. The computation of doubling time (dt) showed values of 9.81±0.001 hours and 10.51±0.01 hours at 30° C. and 37° C., respectively, while the value for 50° C. was higher (14.73±0.02 hours). This further illustrates that this strain grows less efficiently at 50° C. The quantification of remaining CRN following the in-vitro culture demonstrates that a higher rate of degradation occurred at 30° C. followed by 37° C., and finally by 50° C., with the percentage of degradation being approximately 50%, 35%, and 33%, respectively, as shown in FIG. 3B. These differences were not statistically significant (p>0.05); however, a temperature of 30° C. appeared to be associated with a higher rate of CRN degradation (50% at 30° C. compared to 35% at 37° C.). Further, at 50° C. (associated with low bacterial growth), the degradation reached 33%. Thus, this species of bacteria has a relatively wide temperate range for growth and CRN degradation. This is consistent with previous reports on the ability of different Halomonas species to grow at a wide range of temperatures, including strains belonging to species such as Halomonas axialensis, Halomonas hydrothermalis, Halomonas neptunia, and Halomonas sulfidaeris [Kaye, J. Z., Márquez, M. C., Ventosa, A. & Baross, J. A. (2004). Halomonas neptunia sp. nov., Halomonas sulfidaeris sp. nov., Halomonas axialensis sp. nov. and Halomonas hydrothermalis sp. nov.: halophilic bacteria isolated from deep-sea hydrothermal—vent environments, which is incorporated herein by reference in its entirety].

### Example 13: pH Effect

In addition to pH 7, further bacterial growth was assessed at pH 3 and pH 10. These experiments were performed at 37° C., a concentration of 16.6 UM of coronene, and 10% NaCl (w/v). No growth was observed at the acidic pH 3 or the alkaline pH 10. Generally, the desired pH range for PAH degradation falls between pH 6 and pH 8, with neutral pH (pH 7) being the most common [Leahy, J. G. & Colwell, R. R. (1990). Microbial degradation of hydrocarbons in the environment. Microbiol Rev 54, pp. 305-315, which is incorporated herein by reference in its entirety]. PAH biodegradation has also been reported under extreme pH conditions. For instance, pyrene degradation has been reported at pH 9 by the alkaliphilic Mycobacterium sp. strain MHP-1, and the degradation of naphthalene and phenanthrene has been reported in acidophilic conditions as low as pH 3. Bacteria belonging to the genera Clavibacter, Arthrobacter, and Acidocella have also been reported to grow in the presence of naphthalene at a pH as low as 3.5 [Dore, S. Y., Clancy, Q. E., Rylee, S. M. & Kulpa Jr., C. F. (2003). Naphthalene-utilizing and mercury-resistant bacteria isolated from an acidic environment. Appl Microbiol Biotechnol, which is incorporated herein by reference in its entirety]. Bacterial degradation of PAHs has also been reported in high alkaline or acidophile conditions by bacteria belonging to genera such as Marinobacter, Pseudomonas, and Stappia. In relation to the Halomonas genus bacteria, pyrene degradation at pH 7 and pH 9 has been reported in H. shengliensis and H. smyrnensis and salinity at 10% and 20%, respectively. The strain isolated in this study could not grow at either high or low pH, such as pH 10 or 3; however, it may not be ruled out that this strain may grow at moderate-high or moderate-low pHs, such as pH 7 to pH 9 or pH 7 to pH 4.

### Example 14: Salinity Effect

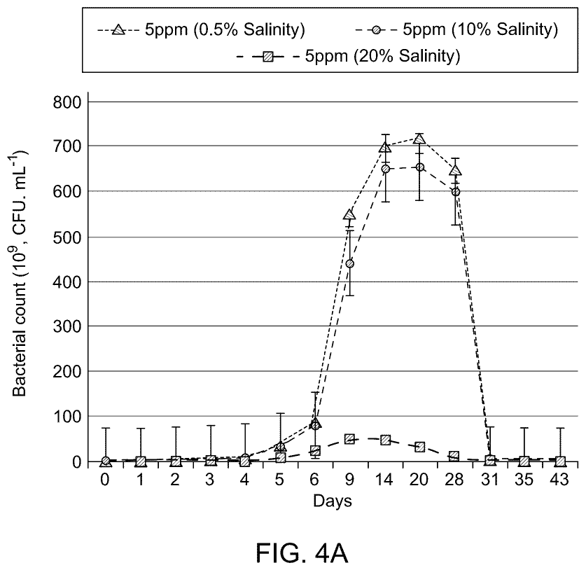

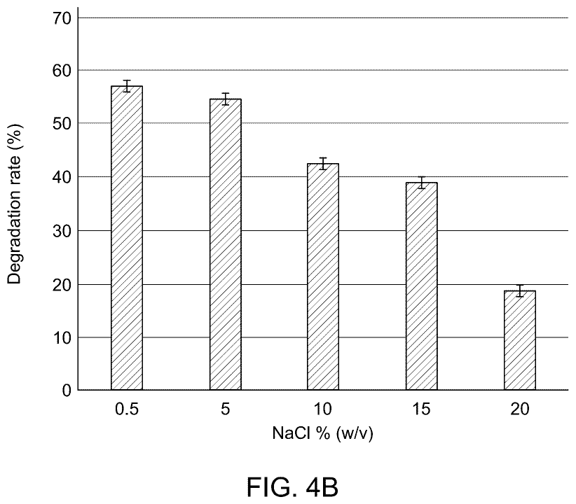

The effect of salinity on bacterial growth was assessed at 0.5%, 10%, and 20% NaCl, while the concentration, pH, and temperature were fixed at 16.6 μM, 7, and 37° C., respectively. As shown in FIG. 4A, growth profiles at 0.5% and 10% NaCl were similar, with the maximum growth attained at day 20 (about 7.10¹¹ CFU mL-1), and the corresponding dt values were 9.86±0.004 hours and 10.42±0.001 hours for 0.5% and 10% NaCl, respectively. The bacterial growth was reduced at a salinity of 20%, with a maximum growth of around 10¹¹, within 9 days, and a dt of 11.78±0.01 hours. The higher ability of this strain to grow at 0.5% is confirmed by the quantification of the remaining CRN in vitro, which shows a higher rate of degradation of 57% at 0.5% NaCl. This rate decreases as the salinity increases to 5%, 10%, 15%, and 20% NaCl, as shown in FIG. 4B. aAt 20% NaCl, the highest salinity, the degradation rate was around 19%, indicating that bacteria were still active at this salinity level, as shown in FIG. 4B. The ANOVA test indicated these differences were not statistically significant (p>0.05). The data suggests that this strain is active within a wide range of salt concentrations, from 0.5% to 20%. Similar results were reported in the degradation of another PAH, pyrene, in Halomonas strains.

### Example 14: Effect of Time Degradation

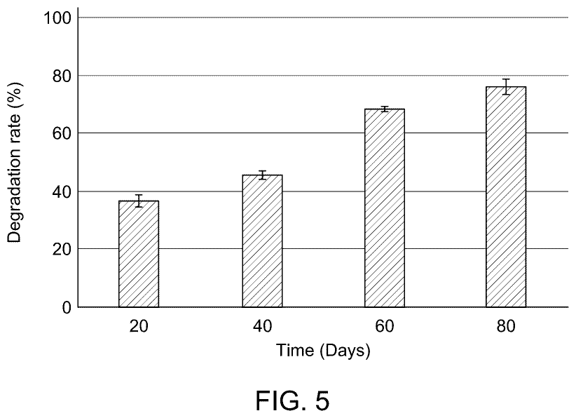

The data discussed in all the aforementioned experiments were obtained within 30 days. Since degradation increases with time, the extent of degradation over 80 days was assessed. This investigation was carried out at 37° C., with a salinity of 10% NaCl, pH 7, and a CRN concentration of 16.6 μM. As can be seen from FIG. 5, at day 20, the degradation rate was around 37%, steadily increasing over time. At day 80, corresponding to the end of the experiment, almost 76% of CRN was degraded. These values of degradation rates were statistically different (ANOVA test, p<0.00001), and the trend analysis indicated a linear model of: degradation (%)=0.7*days+21.4 (R²=0.96, p-value<0.000001), giving rise to a rate of degradation of 0.116 μM CRN day⁻¹ (0.7% of 16.6 μM per day). This is the first report on the degradation rate of CRN in a bacterial cell, so these results may not be compared with any reported ones. The closest comparison may be made with the five-ring PAH benzo[a]pyrene, for which the reported degradation rates in various bacterial strains fell within 0.04-0.3 μM day-1 [Nzila, A., Musa, M. M., Sankara, S., Al-Momani, M., Xiang, L. & Li, Q. X. (2021). Degradation of benzo[a]pyrene by halophilic bacterial strain Staphylococcus haemoliticus strain 10SBZ1A, which is incorporated herein by reference in its entirety]. The rate reported in the current work, 0.1162 μM per day, falls within this range. Thus, this Halomonas caseinilytica, 10SCRN4D strain may degrade the seven-ring PAH CRN as efficiently as other bacterial strains degrade benzo[a]pyrene.

### Example 15: Biodegradation of PAHs of Lower Molecular Weights

The ability of 10SCRN4D to degrade small PAHs, such as benzo[a]pyene, pyrene, and phenanthrene, was investigated. The experiments were conducted at 37° C., a pH of 7, and 10% NaCl for 30 days in the presence of 20 μM of each of the tested PAHs, and PAH degradation was quantified using GC. Under these conditions, the 10SCRN4D strain degraded 43.68%±19.36%, 51.75%±13.67%, and 41.83%±16.24% of benzo[a]pyene, pyrene, and phenanthrene, respectively.

Bacteria gradually break down PAHs by initial oxidation, followed by ring opening, resulting in PAHs with lower molecular weights and, eventually, monoaromatic rings and aliphatic derivatives. Bacteria that can degrade high molecular weight PAHs may also degrade lower molecular weight PAHs. For example, bacterial strains belonging to the genus Ochrobactrum, Cellulosimicrobium, Hydrogenophaga, Rhizobium tropici, and Staphylococcus, selected for their ability to degrade benzo[a]pyrene, have also been shown to degrade other PAHs of lower molecular weight such as pyrene, phenanthrene, anthracene, and naphthalene.

The present disclosure provides a halophilic bacterial strain, Halomonas caseinilytica, 10SCRN4D, capable of degrading coronene when used as a sole source of carbon, which has been isolated and characterized for the first time. The strain may degrade coronene at a rate as high as those reported for lower molecular-weight PAHs. In addition, it may degrade coronene within a wide range of salinity (0.5%-20% NaCl), making it a beneficial bacterial strain to be used in the context of bioremediation of an environment contaminated with coronene.

Numerous modifications and variations of the present disclosure are possible in light of the above teachings. It is therefore to be understood that within the scope of the appended claims, the disclosure may be practiced otherwise than as specifically described herein.

1: A method for removing polycyclic aromatic hydrocarbons from a sample, comprising:
contacting the sample with a bacterial strain for a time sufficient to degrade at least one polycyclic aromatic hydrocarbon,
wherein the sample comprises coronene at a first concentration,
wherein the bacterial strain is Halomonas caseinilytica,
degrading the at least one polycyclic aromatic hydrocarbon; and
collecting a product containing coronene at a second concentration lower than the first concentration.

2: The method of claim 1, wherein the bacterial strain is Halomonas caseinilytica, 10SCRN4D.

3: The method of claim 2, wherein bacteria of the bacterial strain are rod-shaped with an average length of 1.0 to 3.0 micrometer (μm) and an average width of 0.1 to 1.0 μm.

4: The method of claim 1, wherein the sample is contacted with a bacterial strain for from 1 to 100 days.

5: The method of claim 1, wherein the first concentration is from 10 to 200 micromolar (μM).

6: The method of claim 1, wherein the second concentration is from 20 to 75 percent lower than the first concentration based on the total weight of the sample and the total weight of the coronene before and after the degrading.

7: The method of claim 1, wherein a salinity of the sample is from 0.1 to 25 percent weight sodium chloride by volume based on a total volume of the sample.

8: The method of claim 1, wherein the contacting occurs at a temperature from 25 to 50° C.

9: The method of claim 1, wherein the contacting occurs at a pH of 4 to 9.

10: The method of claim 1, wherein the contacting occurs for 78 to 82 days, and a rate of degradation is 0.100 to 0.130 μM of coronene per day.

11: The method of claim 1, wherein during the contacting the Halomonas caseinilytica reproduces at a doubling rate of 8 to 16 hours.

12: The method of claim 1, wherein the first concentration is 15 to 20 μM, wherein the sample has a pH of 6.8 to 7.2, a temperature of 35 to 40° C., a salinity of 8 to 12 percent weight sodium chloride by volume based on a total volume of the sample, wherein the contacting occurs for 18 to 22 days, and a rate of biodegradation of the coronene is 45 to 50 percent based on an initial concentration of the coronene.

13: The method of claim 12, wherein a starting aliquot of the bacterial strain is 4.5×10⁵ to 5.5×10⁵ colony forming units (CFUs) per milliliter (CFU/mL).

14: The method of claim 13, wherein a maximum count of the bacterial strain is 6×10¹¹ to 8×10¹¹ CFU/mL.

15: The method of claim 1, wherein the first concentration is 15 to 20 μM, wherein the sample has a salinity is 0.2 to 0.8 percent weight sodium chloride by volume based on a total volume of the sample, a pH of 6.8 to 7.2, a temperature of 35 to 40° C., and a rate of biodegradation of the coronene is 55 to 60 percent based on an initial concentration of the coronene.

16: The method of claim 1, wherein the first concentration is 15 to 20 μM, wherein the sample has a temperature of 28 to 32° C., a pH of 6.8 to 7.2, a salinity of 8 to 12 percent weight sodium chloride by volume based on a total volume of the sample, and a rate of biodegradation of the coronene is 40 to 60 percent based on an initial concentration of the coronene.

17: The method of claim 1, wherein the sample further comprises one or more polycyclic aromatic hydrocarbons selected from the group consisting of benzo[a]pyrene, pyrene, and phenanthrene.

18: The method of claim 17, wherein the polycyclic aromatic hydrocarbon is benzo[a]pyrene and 20 to 65 percent of the polycyclic aromatic hydrocarbon is degraded based on an initial amount of the benzo[a]pyrene.

19: 19: The method of claim 17, wherein the polycyclic aromatic hydrocarbon is pyrene and 35 to 70 percent of the polycyclic aromatic hydrocarbon is degraded based on an initial amount of the pyrene.

20: The method of claim 17, wherein the polycyclic aromatic hydrocarbon is phenanthrene and 20 to 60 percent of the polycyclic aromatic hydrocarbon is degraded based on an initial amount of the phenanthrene.
